(19) United States
(12) Patent Application Publication
(10) Pub. No.: US 2007/0043445 A1
(43) Pub. Date: Feb. 22, 2007

(54) Movable neck and stem for hip prosthesis, made in such a way as to facilitate their disengagement

(76) Inventor: Sheila Ragbir, Whim (TT)

Correspondence Address: YOUNG & THOMPSON, 745 SOUTH 23RD STREET, 2ND FLOOR, ARLINGTON, VA, 22202, US

(21) Appl. No.: 10/547,149

(22) PCT Filed: Dec. 8, 2004
(86) PCT No.: PCT/IB04/04074

§ 371 Date: Aug. 26, 2005

(51) Int. Cl. A61F 2/36

(52) U.S. Cl. 623/22.12; 623/22.46

(57) ABSTRACT

What is described is an assembly consisting of a movable neck and a stem for a hip prosthesis, the said movable neck being provided with a tapered part connected in a complementary way to a cavity formed in the said stem. In the assembly according to the invention, a through hole is formed in the movable neck or in the stem, this hole being orientated and positioned so that, when the tip of a pointed tool with its outer walls converging towards the tip, the opposite sides of these walls bear on a surface forming part of the movable neck and a surface forming part of the stem, exerting, by the inclined plane effect, a substantially axial force capable of causing the disengagement of the movable neck from the stem.

The present invention relates to the technical field of hip prostheses, and more particularly to an assembly consisting of a movable neck and a stem (in other words the part which is inserted into the femur).

As is known to those skilled in the art, a movable neck for a hip prosthesis terminates in its lower area in a tapered part which is inserted by a forced connection into a cavity having a substantially complementary shape formed in the upper end of the stem.

The assembly described above, consisting of a movable neck and stem separate from each other, has recently replaced the preceding system known as a “one-piece system”, in which the neck and stem formed a single unit, since it offers numerous advantages, including the possibility of fitting an anatomically matching neck and of replacing it easily, both during the operation for fitting the prosthesis and on subsequent occasions, without the need to remove the stem from its housing formed in the femur.

However, those working in the field of hip prosthesis surgery have discovered that sometimes, when an operation is carried out on a prosthesis used for a certain period of time, the movable neck remains “welded” in the cavity of the stem, as a result of the user's weight, which tends to force the parts more closely together during walking, or as a result of the connective tissue generated by the body after the fitting of the prosthesis, which tends to block even the most minute cavities, causing greater adhesion between the separate parts of the said prosthesis.

This causes objective problems in the operation of detaching the movable neck from the stem, which cannot always be overcome by the use of the appropriate known extractors, which, mutatis mutandis, act on a similar principle to the corkscrew, partly because of the fact that the different inclinations of the necks required by the different anatomies of the femur prevent the extractor from working axially.

The inventor of the proposal of the present invention, which is intended to provide a solution to this problem, has devised an assembly consisting of a movable neck and a stem, having technical characteristics which facilitate the operation of detaching and disengaging the aforesaid two parts from each other.

The inventor considered that, in view of the size of the forces required to cause the aforesaid detachment and their direction parallel to the axis of the said cavity of the stem, it would be possible to use the inclined plane system, which is well known precisely because of the size of the forces which it can generate, if it were possible to apply a pointed tool with converging walls between two surfaces substantially perpendicular to the axis of the aforesaid cavity, one of these surfaces forming part of the movable neck and the other forming part of the stem.

The inventor then had the idea of forming a hole, positioned as described more fully below, perpendicular to the axis of the said cavity and having the characteristics described above.

This idea resulted in two possible embodiments, in which the hole for the introduction of the aforesaid pointed tool is formed in the body of the movable neck, in a position matching the outer surface of the stem with respect to the femur, or in the stem, at the position of the aforesaid cavity and in the proximity of the lower surface of the movable neck located within the said cavity.

The object of the present invention is therefore a movable neck and stem assembly for a hip prosthesis as described in the attached claim 1.

A more detailed description will now be given of two preferred embodiments, with additional reference to the attached drawings, in which:

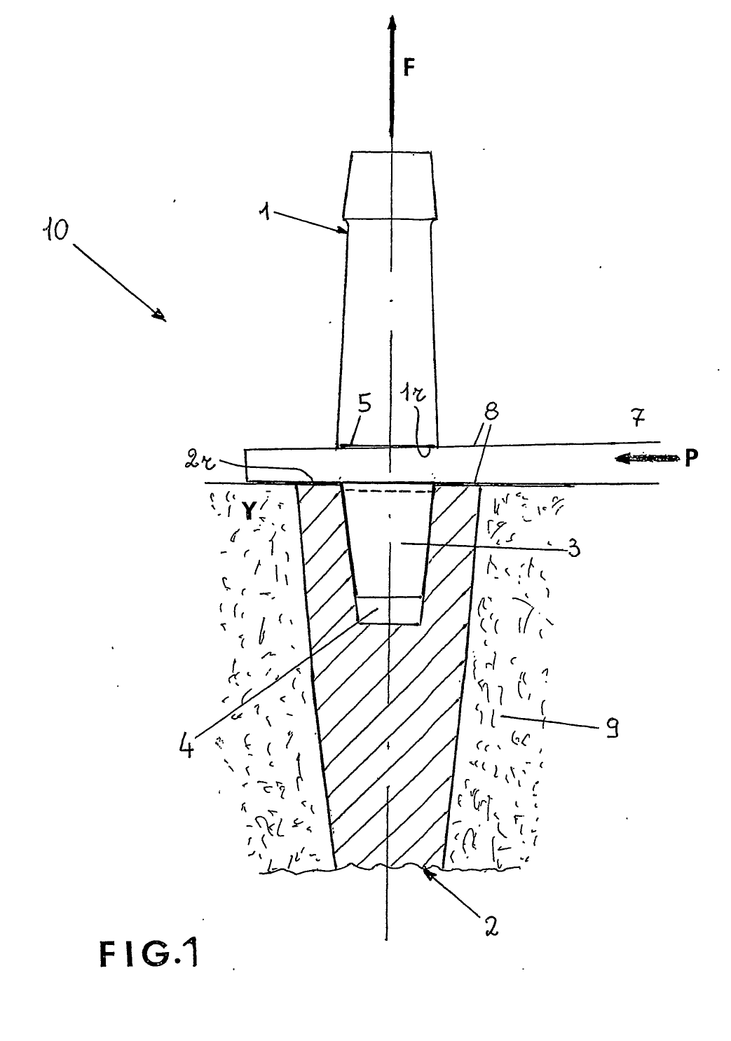

FIG. 1 is a longitudinal section through an assembly according to the invention in which the said through hole for the insertion of the pointed tool for the extraction is formed perpendicularly in the body of the movable neck;

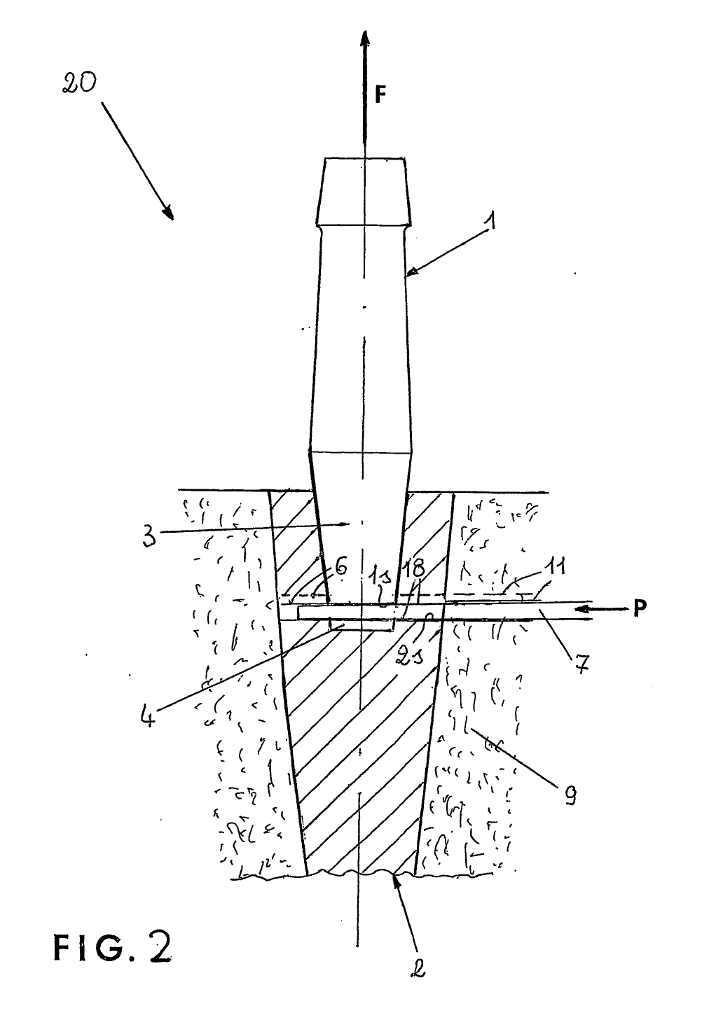

FIG. 2 is a longitudinal section through another assembly according to the invention, in which the said through hole for the insertion of the pointed tool for the extraction is formed perpendicularly in the stem at the position of its cavity.

With reference to FIG. 1, this shows an assembly 10 according to the invention, in which a movable neck 1 is fitted to a stem 2 by means of a tapered part 3 inserted with a forced connection into an approximately complementary cavity 4 formed in the said stem 2.

This stem, as is known, is in turn inserted into the patient's femur 9.

A through hole 5, designed and positioned (solid lines) so that its lower part touches the outer surface 2r of the stem 2, or (broken lines) is partially contained within the cavity 4 of the stem 2, is formed in the body of the movable neck 1, transversely and perpendicularly to the latter.

Clearly, when the tip of a pointed tool 7 having its outer walls 8 converging towards this tip is inserted coaxially into the hole 5, and a force P is exerted on the pointed tool, the said walls 8 bearing on opposite sides against the said outer surface 2r of the stem 2 and against the upper wall 1r of the said hole (5) forming part of the movable neck 1. Therefore, an increase in the intensity of the force P generates, according to the known principles of the inclined plane, a force F, coaxial with the cavity 4 of the stem 2, which causes the extraction of the movable neck 1.

FIG. 2 shows another example of embodiment 20 of the assembly according to the invention. In this example also, the movable neck 1 has its tapered part 3 inserted into the cavity 4 of the stem 2, which in turn is housed in the femur 9.

In this case, however, the said through hole 6 is formed in the stem 2 perpendicularly to the cavity 4, and is designed and positioned so that touches with its upper part (solid lines) the lower surface is of the stem 1, or (broken lines) so that it faces and is perpendicular to this surface.

As in the preceding case, if the tip of the previously described pointed tool 7 is inserted into the hole 6 and a force P is exerted on it, its converging opposite walls 18 bear on the wall 2s of the hole 6 forming part of the stem 2 and on the lower wall 1s of the movable neck 1, so that, when the intensity of the force S is increased, an extraction force F is generated, this force being parallel to the axis of the cavity 4 and sufficient to cause the extraction of the movable neck 1 from the stem 2.

Clearly, in the last case described, a hole 11 must also be made in the walls of the femur 9 to permit the passage of the pointed tool 7.

In both cases, the invention provides a system in which a correctly orientated force of considerable intensity is easily exerted, causing the disengagement of a movable neck 1 from the cavity 4 of a stem 2, thus achieving the inventor's object.

It should be noted that the holes 5 and 6 described above can have a circular or slotted cross section, according to design requirements. To enable pointed tools 7 with different transverse dimensions to be used, the inventor suggests that slotted holes be used in most cases.

As regards the said pointed tool 7, its cross section can be polygonal or circular.

1. Assembly (10, 20) consisting of a movable neck (1) and a stem (2) for a hip prosthesis, the said movable neck (1) being provided with a tapered part (3) connected in a complementary way to a cavity (4) formed in the said stem (2), characterized in that a through hole (5, 6) is formed in the movable neck (1) or in the stem (2), this hole being orientated and positioned in such a way that, when the tip of a pointed tool (7) with its outer walls (8) converging towards this tip is inserted coaxially into the hole, these walls bear on opposite sides against a surface (1r, 1s) forming part of the movable neck (1) and a surface (2r, 2s) forming part of the stem (2), thus exerting, by the inclined plane effect, a substantially axial force sufficient to cause the extraction of the movable neck (1) from the stem (2).

2. Assembly according to claim 1, characterized in that the said through hole (5) is formed transversely in the body of the movable neck (1), and is positioned and designed in such a way that its lower part touches the surface (2r) of the outer stem (2) with respect to the femur (9) into which it is inserted or partially contained in the said cavity (4) of the said stem (2).

3. Assembly according to claim 1, characterized in that the said through hole (6) is formed transversely in the stem (2), and is positioned and designed in such a way as to touch the lower surface (1s) of the movable neck (1) or to be perpendicular to it and facing it.

4. Assembly according to claim 1, in which the said pointed tool (7) has a circular or polygonal cross section.

5. Assembly according to claim 2, in which the said pointed tool (7) has a circular or polygonal cross section.

6. Assembly according to claim 3, in which the said pointed tool (7) has a circular or polygonal cross section.
